(19) United States
(12) Patent Application Publication
(10) Pub. No.: US 2006/0064031 A1
(43) Pub. Date: Mar. 23, 2006

(54) Biopsy needle

(76) Inventor: Stuart H. Miller, Clifton, NJ (US)

Correspondence Address: WELSH & FLAXMAN LLC, 2000 DUKE STREET, SUITE 100, ALEXANDRIA, VA, 22314, US

(21) Appl. No.: 11/228,466

(22) Filed: Sep. 19, 2005

Related U.S. Application Data
(60) Provisional application No. 60/610,542, filed on Sep. 17, 2004.

(51) Int. Cl. A61B 10/00

(52) U.S. Cl. 600/567; 600/564

(57) ABSTRACT

A biopsy needle includes an outer cannula and an inner cannula. The inner cannula includes a distal end and a proximal end. The outer cannula is shaped and dimensioned to closely circumscribe the inner cannula for movement relative thereto. The inner cannula further includes a sample recess at its distal end, the sample recess including a U-shaped cutting edge defined by
pₙ=xₙ cos θ
qₙ=xₙ sin θ where, θ is an angle at which the cutting edge is formed.
xₙ is an actual length position along the sample recess,
pₙ is a length position of the sample recess relative to the cutting edge along a line substantially parallel to an inner cannula longitudinal axis, and
qₙ is a depth position of the sample recess relative to an apex of an arc defined by a cutting edge.

### CROSS REFERENCE TO RELATED APPLICATIONS

This application is based upon U.S. Provisional Application Ser. No. 60/610,542, entitled “BIOPSY NEEDLE”, which was filed Sep. 17, 2004.

### BACKGROUND OF THE INVENTION

1. Field of the Invention

The invention relates to a biopsy needle. More particularly, the invention relates to a biopsy needle having a sample recess shaped and dimensioned to optimize operation of the biopsy needle. The invention also relates to a method for forming the recess.

2. Description of the Prior Art

Biopsy needles are currently available in gages ranging from 14 to 20. The biopsy samples are obtained using various methods. One of the commercially available designs employs a solid, pointed cannula inside of a slip-fitted outer cannula with a beveled leading end. The solid inner cannula contains a rectangular cavity, which collects the biopsy sample. The biopsy sample is removed from the tissue by inserting the biopsy needle into the tissue, uncovering the collection portion, that is, the rectangular cavity, and firing the outer cannula forward quickly using a spring. The portion of the tissue in the region of the sample collection rectangular cavity is torn from the surrounding tissue and collected in the rectangular cavity. Biopsy sample collection using this “brute force” approach results in trauma to the patient. A need exists, therefore, for a biopsy needle, that obtains the desired sample by cutting rather than tearing the sample from the surrounding tissue.

### SUMMARY OF THE INVENTION

It is, therefore, an object of the present invention to provide a biopsy needle including an outer cannula and an inner cannula. The inner cannula includes a distal end and a proximal end. The outer cannula is shaped and dimensioned to closely circumscribe the inner cannula for movement relative thereto. The inner cannula further includes a sample recess at its distal end, the sample recess including a U-shaped cutting edge defined by
pₙ=xₙ cos θ
q_{N}=xₙ sin θ
where,
   θ is an angle at which the cutting edge is formed.
   xₙ is an actual length position along the sample recess,
   pₙ is a length position of the sample recess relative to the cutting edge along a line substantially parallel to an inner cannula longitudinal axis, and
   qₙ is a depth position of the sample recess relative to an apex of an arc defined by a cutting edge.

It is also an object of the present invention to provide an inner cannula of a biopsy needle manufactured in accordance with the method comprising setting a grinding wheel at an angle θ, relative to a longitudinal axis of the inner cannula and grinding the inner cannula with the grinding wheel to produce a sample recess having a cutting edge at an outside surface of the inner cannula as the grinding wheel moves through the inner cannula in a direction transverse to the longitudinal axis of the inner cannula. The cutting edge is defined as above.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a side view of the present biopsy needle.

FIGS. 2 and 3 are cross sectional views of the biopsy needle showing operation thereof

FIG. 4 is a detailed cross sectional view of the inner cannula about a plane symmetrically bisecting the sample recess.

FIG. 5 is a detailed cross sectional view of the biopsy needle about a plane symmetrically bisecting the sample recess.

FIG. 6 is a cross sectional view along the line VI-VI in FIG. 5.

FIG. 7 is a top view of the inner cannula.

FIGS. 8 through 15 are various studies of the biopsy needle in accordance with the present invention.

FIG. 16 is a cross sectional view in accordance with an alternate embodiment.

FIG. 17 is a cross sectional view in accordance with yet a further alternate embodiment.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

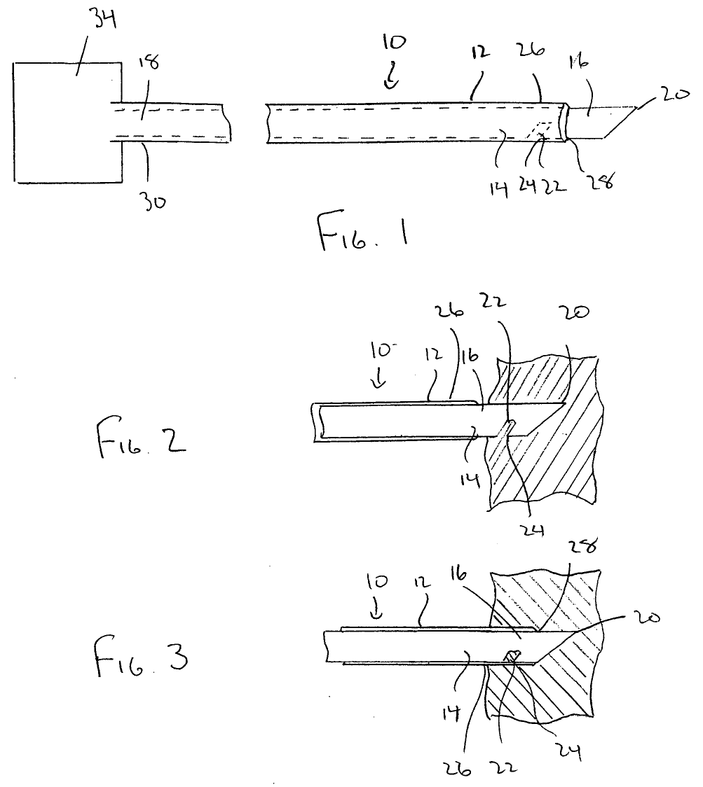

With reference to FIGS. 1 through 7, a biopsy needle 10 in accordance with the present invention is disclosed. The biopsy needle 10 generally includes a cutting outer cannula 12 that surrounds a solid inner cannula 14. As those skilled in the art will certainly appreciate, the cutting outer cannula 12 is shaped and dimensioned to fit about the inner cannula 14 in a manner permitting relative movement with the removal of core biopsy samples upon proper actuation of the biopsy needle 10. With this in mind, the outer cannula 12 fits closely about the inner cannula 14 to facilitate cutting of the sample tissue as the outer cannula 12 is moved relative to the inner cannula 14; that is, the outer cannula 12 is a slip-fit over the inner cannula 14.

In general, the inner cannula 14 includes a distal end 16 and a proximal end 18. The distal end 16 is provided with a distal tip 20 and a sample recess 22 having a cutting edge 24. The distal tip 20 is a traditional point tip adapted to easily move through tissue with the creation of limited trauma.

As to the outer cannula 12, it also includes a distal end 26 having a sharpened distal tip 28 and a proximal end 30. The outer cannula 12 has a beveled, 360-degree cutting edge 32 at its distal tip 28. The leading, cutting edge 32 of the outer cannula 12 is beveled for a full 360 degrees. The outer cannula 12 is suitably spring driven to slide axially along the inner cannula 14 when triggered by the physician. The cutting edges 24, 32 of the inner cannula 14 and outer cannula 12 remove the biopsy sample by cutting the sample in scissor-like fashion as the spring-driven, outer cannula 12 slides axially along the inner cannula 14.

The proximal end 18 of the inner cannula 14 and the proximal end 30 of the outer cannula 12 are respectively coupled to an actuation mechanism 34 controlling movement of the outer cannula 12 relative to the inner cannula 14. In accordance with a preferred embodiment, the actuation mechanism 34 is a spring biased actuation mechanism as disclosed in U.S. Pat. No. 5,425,376 to Banys et al., which is incorporated herein by reference, although other actuation mechanisms may certainly be used without departing from the spirit of the present invention.

The biopsy needle 10 is preferably made of medical grade stainless steel although those skilled in the art will appreciate that it may be manufactured from other materials without departing from the spirit of the present invention.

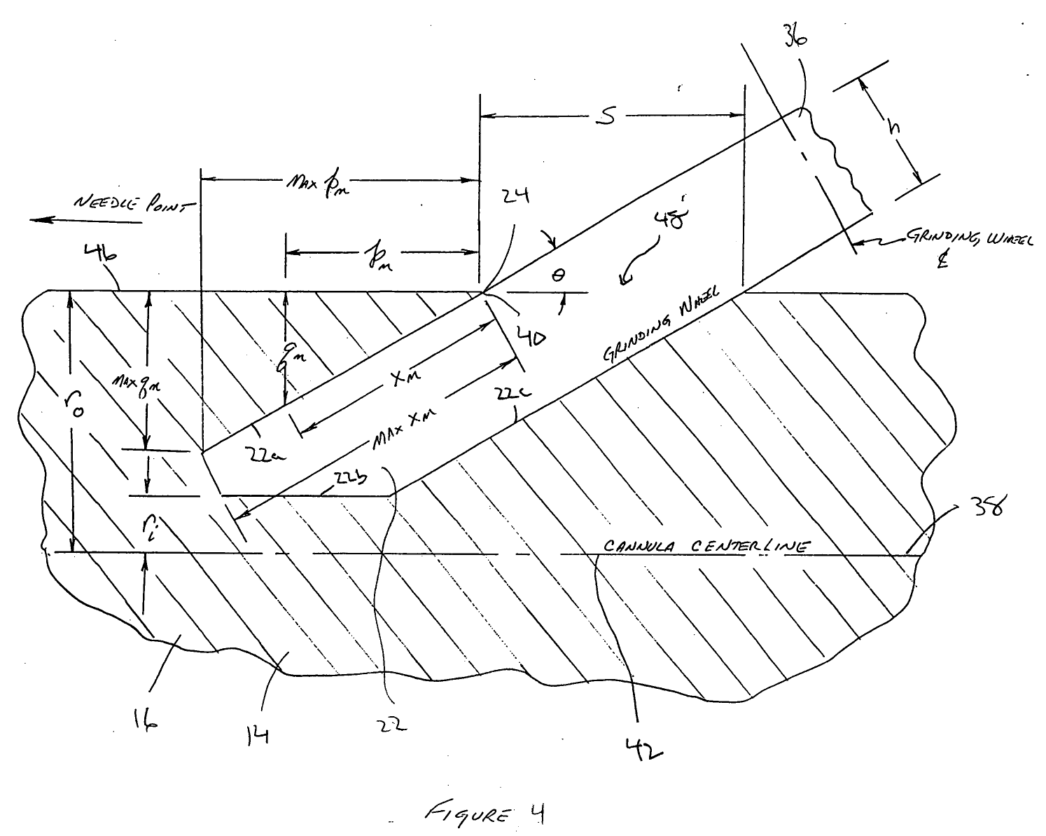

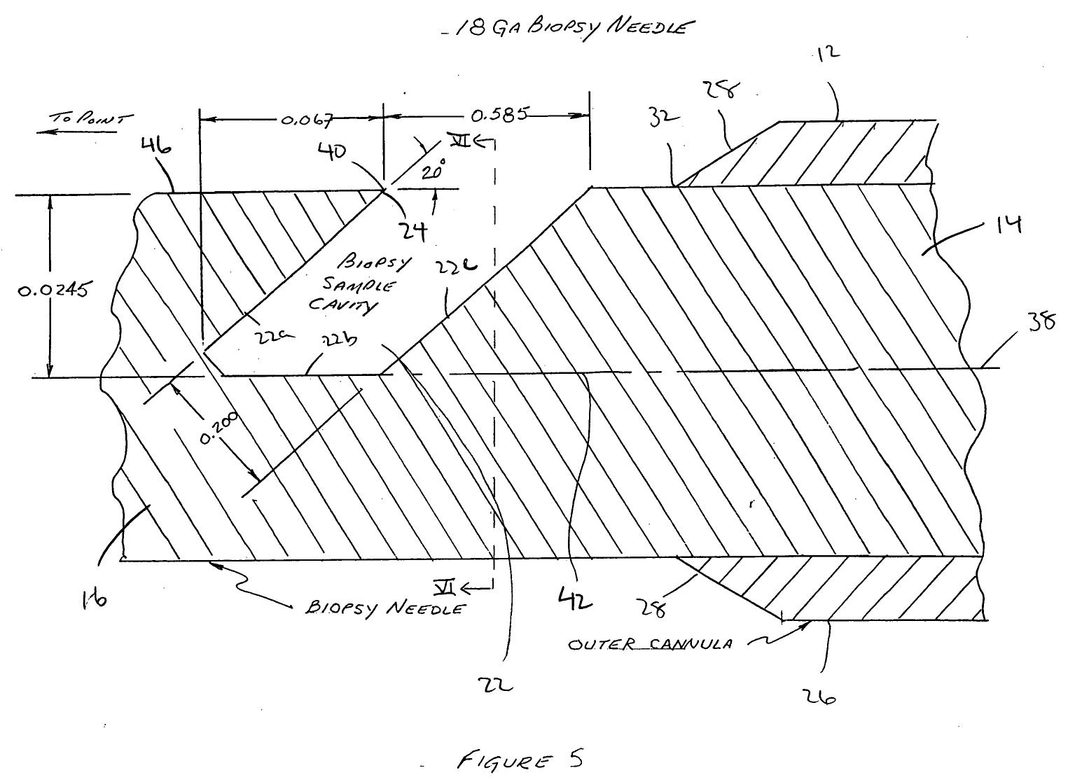

In use, and with reference to FIGS. 2 and 3, the biopsy needle 10 is positioned at a predetermined location where it is desired to obtain a biopsy sample from a patient. The distal end 16 of the inner cannula 14 is exposed with the small sample recess 22 exposed for positioning of a biopsy sample therein. Because of the resilience of the tissue at the predetermined location, a small portion of tissue is forced within the sample recess 22. The outer cannula 12 may then be actuated for movement toward the distal end 16 of the inner cannula 14 in a manner which cuts the tissue such that a small sample is retained within the sample recess 22. With the tissue maintained in the sample recess 22 between the inner surface of the outer cannula 12 and the outer surface of the inner cannula 14, the biopsy needle 10 may be removed from the patient for retrieval of the tissue sample maintained in the sample recess 22.

As briefly discussed above, the sample recess 22 is formed at the distal end 16 of the inner cannula 14. The sample recess 22 includes a forward wall 22a, a base 22b and a rearward wall 22c. The forward wall 22a includes a three dimensional, integral, biopsy sample cutting edge 24 which faces 180 degrees from the distal tip 20 of the inner cannula 14.

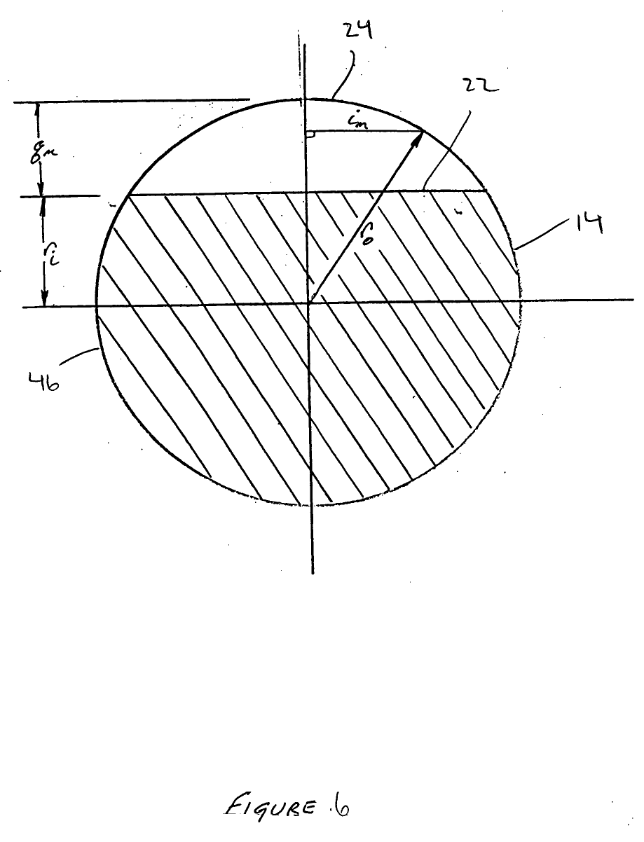

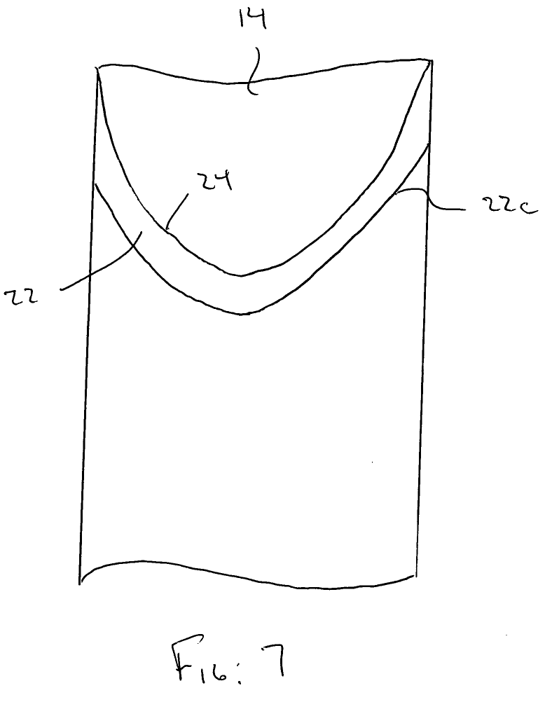

Referring to FIGS. 4 and 5, the method for producing the inner cannula 14 cutting edge 24 on the biopsy needle 10 is shown. The cutting edge 24 is located a short distance behind the distal tip 20 of the inner cannula 14. The cutting edge 24 is produced by grinding the biopsy needle inner cannula 14 with a circular grinding wheel 36, which has been contoured to the geometry shown in FIG. 4. The axis of rotation of the grinding wheel 36 is set at a small angle, θ, off the perpendicular to the longitudinal axis 38 of the biopsy needle, and in particular, the inner cannula 14. The grinding process produces the three-dimensional, “U” shaped cutting edge 24 at the outer diameter (OD), or outside surface, 46 of the biopsy needle inner cannula 14 as the grinding wheel 36 moves through the inner cannula 14 in a direction transverse to the longitudinal axis of the biopsy needle 10.

The geometry of the “U” shaped cutting edge 24 depends on the values of the design parameters used. For any selected inner cannula 14 outer diameter (OD), the closed end of the “U” shaped cutting edge 24 can be ground to produce a relatively broad or a relatively sharp point 40 by changing the grinding angle, θ, shown in FIG. 4.

A mathematical analysis was conducted to derive the equations necessary to design the three-dimensional “U” shaped cutting edge 24 for the inner cannula 14 of the biopsy needle 10. FIG. 4 shows the grinding wheel 36 at some arbitrary depth, rᵢ, in the biopsy needle inner cannula 14 wherein rᵢ is the distance from the centerline 42 of the inner cannula 14 to which the grinding wheel 36 penetrates while forming the sample recess 22 (which may also be considered the base 22b of the sample recess 22). The points designated by the parameters xₙ, pₙ, qₙ define the “U” shaped, biopsy needle cutting edge 24 when viewed perpendicular to the longitudinal, transverse axis of the inner cannula 14 (as shown with reference to FIG. 4). By way of explanation xₙ is the actual length position along the sample recess 22, pₙ is the length position of the sample recess 22 relative to the cutting edge 24 along a line substantially parallel to the inner cannula 14 longitudinal axis and qₙ is the depth position of the sample recess 22 relative to the apex of the arc defined by the cutting edge 24.

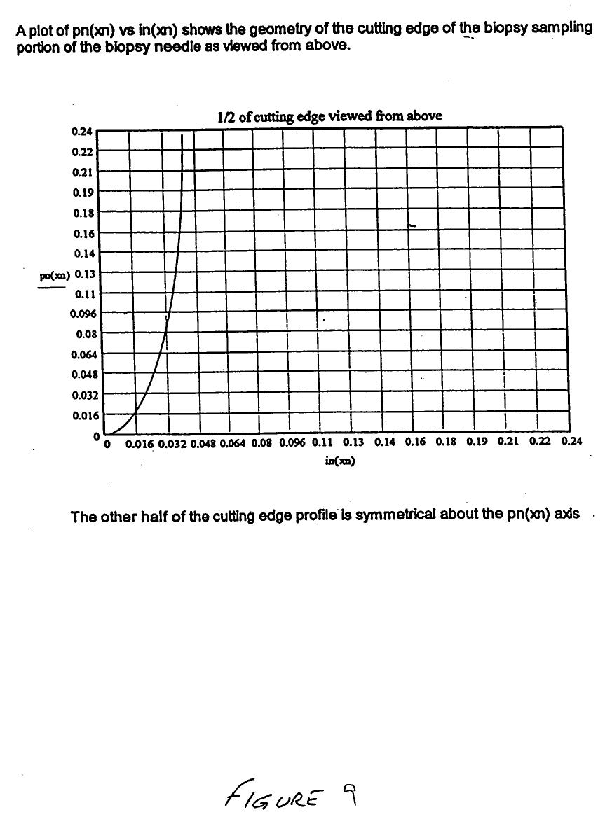

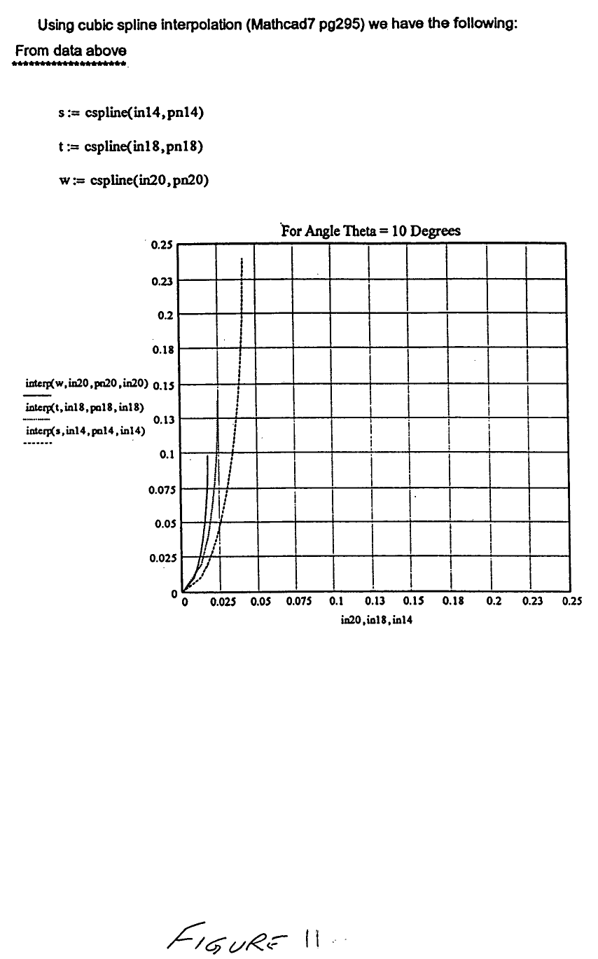

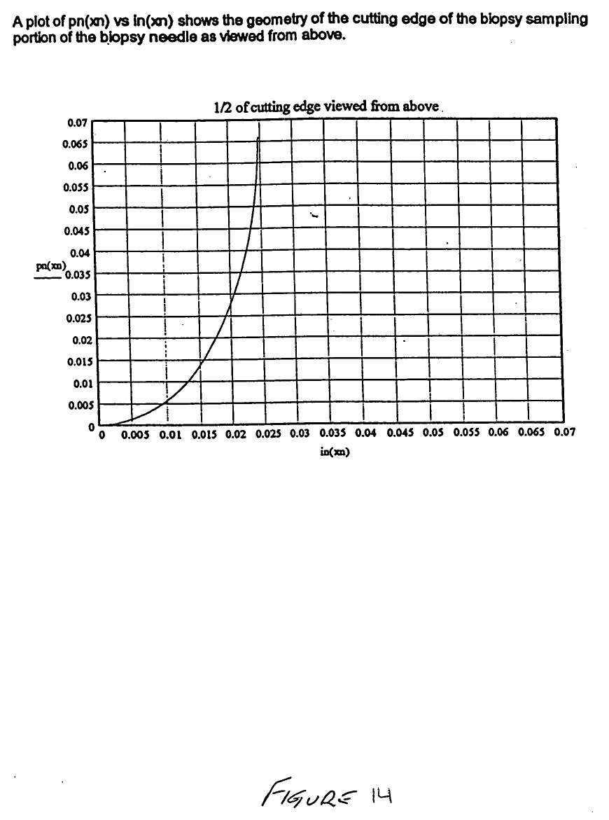

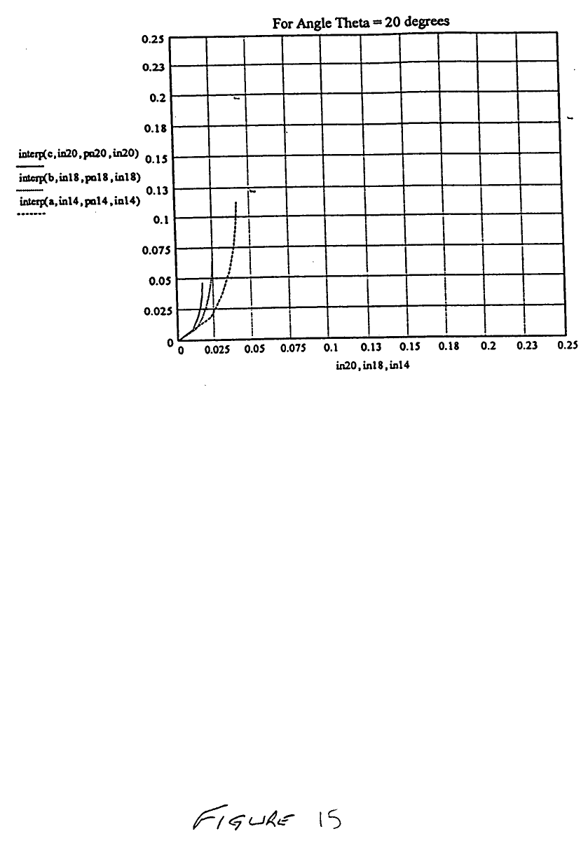

When viewed from the perspective shown in FIG. 4, the “U” shaped profile appears to be merely a taper. However, when viewed from above the inner cannula 14 (see FIGS. 7 through 15), the “U” shaped geometry is clearly evident. The three-dimensional, “U” shaped geometry is, of course, created by the intersection of the flat outer surface of the grinding wheel 36 with the outside surface 46 of the biopsy needle inner cannula 14.

Referring to FIG. 4, 5 and 6, one can write the following equations for pₙ and qₙ in terms of xₙ:
pₙ=xₙ cos θ
qₙ=xₙ sin θ

One can also write the maximum value of xₙ and pₙ as:
max xₙ=rₒ−rᵢ/sin θ
max pₙ=rₒ−rᵢ/tan θ

The approximate, maximum value of qₙ is:
max qₙ=rₒ−rᵢ

Those maximum values represent the values of pₙ, xₙ and qₙ at the base 22b of the sample recess 22 and the outer surface of the grinding wheel 36 forming the sample recess 22.

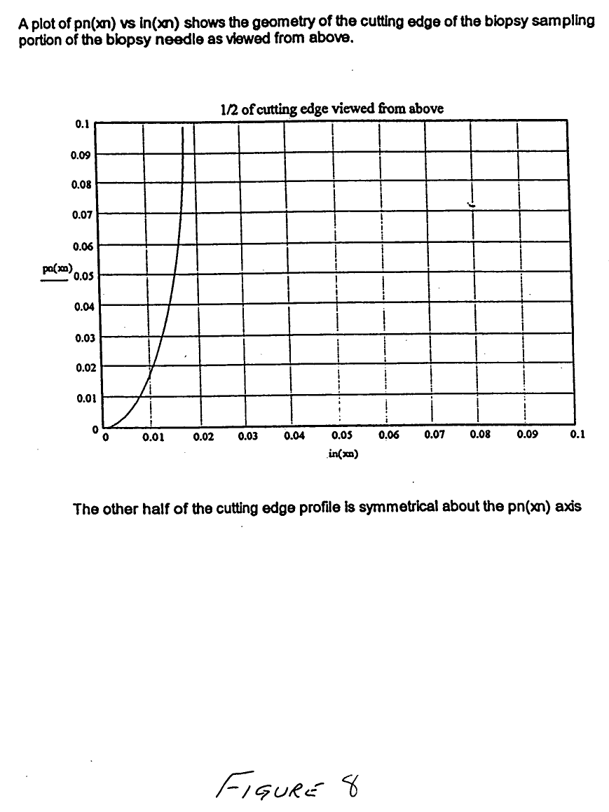

FIG. 6 shows the transverse cross section of the biopsy needle inner cannula 14. The distance iₙ is the distance from the centerline 42 of the transverse cross section of the inner cannula 14 to the point of intersection of the grinding wheel 36 (or the sharp point 40 of the cutting edge 24) and the OD of the inner cannula 14. The value of in can be written:
iₙ±\sqrt{(2r₀−xₙ sin θ)xₙ sin θ}

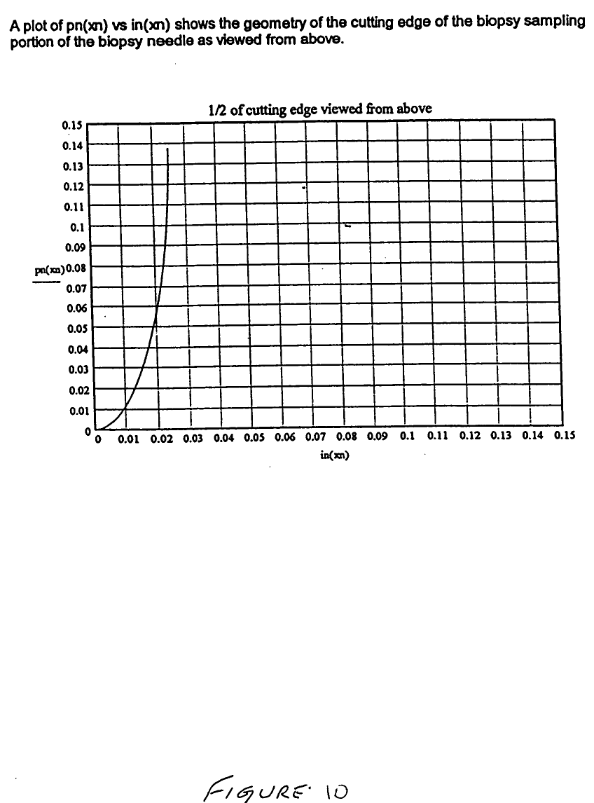

From pₙ, qₙ, and iₙ we can plot the three dimensional geometry of the “U” shaped cutting edge 24 on the inner cannula 14. For our purposes, a two-dimensional plot of pₙ vs. iₙ shows the geometry of the “U” shaped cutting edge when viewed from above the inner cannula 14. The variation in cutting edge geometry is readily apparent from a plot of pₙ vs. iₙ (see FIGS. 8 through 15). The length of the biopsy sample cavity, S, is:
S=h/sin θ
where,
   h is the thickness of the grinding wheel 36 and/or the resulting opening distance 48 of the sample recess 22.

Now that a basic understanding of the sample recess geometry is appreciated, the preferred embodiments which optimize operation in accordance with the present invention are disclosed. To demonstrate the use of the design equations, the geometry of the biopsy needle “U” shaped cutting edge was calculated for three needle gages, namely 20 GA, 18 GA, and 14 GA. The 20 to 14 GA range covers the range of gages currently being used, with particularly heavy usage being noted with regard to the 18 GA size. In the three examples given below, the grinding wheel penetration is approximately to the centerline of the inner cannula. This was accomplished by setting the parameter rᵢ to zero in the equations. The design parameters and the results are listed below:

| Example 1 (20 GA) | Example 2 (14 GA) | Example 3 (18 GA) |
|---|---|---|
| rₒ = 0.0175″ | rₒ = 0.0415″ | rₒ = 0.0245″ |
| rₒ = 0 | rₒ = 0 | rₒ = 0 |
| h = 0.20″ | h = 0.20″ | h = 0.20″ |
| θ = 10° | θ = 10° | θ = 10° |
| max xₙ = 0.1008″ | max xₙ = 0.2390″ | max xₙ = 0.1411″ |
| max pₙ = 0.0992″ | max pₙ = 0.2354″ | max pₙ = 0.1379″ |
| max qₙ = 0.0175″ | max qₙ = 0.0175″ | max qₙ = 0.0175″ |
| max iₙ = ±0.0175″ | max iₙ = ±0.0415″ | max iₙ = ±0.0245″ |

The pₙ vs. iₙ curves for the three examples showing the geometry of the cutting edge when viewed from above the inner cannula of the biopsy needle are given in FIGS. 8, 9 and 10, respectively.

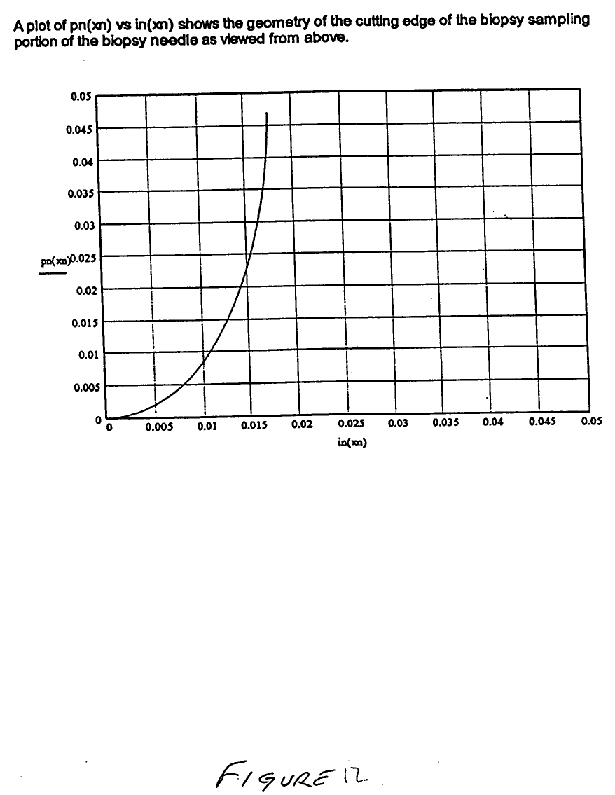

A comparison of the three pₙ vs. iₙ curves is shown in FIG. 11 for θ=10°. The length of the biopsy sample recess for all three examples is 1.151″.

To demonstrate the influence of the angle, θ, in the three examples given above, the value of θ was set equal to 20° in all three examples. The results are as follows:

| Example 1 (20 GA) | Example 2 (14 GA) | Example 3 (18 GA) |
|---|---|---|
| max xₙ = 0.0512″ | max xₙ = 0.1213″ | max xₙ = 0.0716″ |
| max pₙ = 0.0481″ | max pₙ = 0.1140″ | max pₙ = 0.0673″ |
| max qₙ = 0.0175″ | max qₙ = 0.0415″ | max qₙ = 0.0245″ |
| max iₙ = ±0.0175″ | max iₙ = ±0.0415″ | max iₙ = ±0.0245″ |

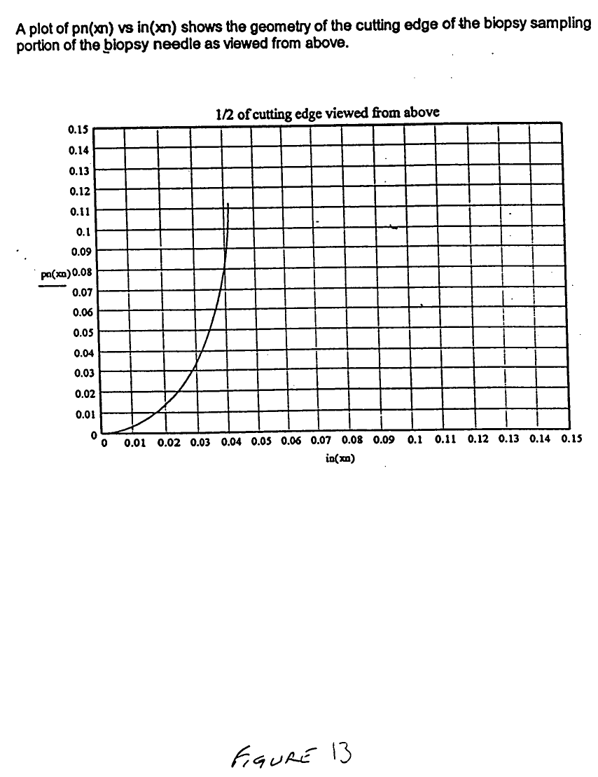

The length of the sample cavity, S, is 0.585″ for all three examples. The pₙ vs. iₙ curves are given in FIGS. 12 through 15.

The results for all of the examples are summarized below:

| GA | θ,° | max xₙ″ | max pₙ″ | max qₙ″ | max iₙ″ | s,″ |
|---|---|---|---|---|---|---|
| 20 | 10 | 0.01008 | 0.0992 | 0.0175 | 0.0175 | 1.151 |
| 20 | 20 | 0.0512 | 0.0481 | 0.0175 | 0.0175 | 0.585 |
| 14 | 10 | 0.2399 | 0.2354 | 0.0145 | 0.0145 | 1.151 |
| 14 | 20 | 0.1213 | 0.1140 | 0.0145 | 0.0145 | 0.585 |
| 18 | 10 | 0.1411 | 0.1389 | 0.0245 | 0.0245 | 1.151 |
| 18 | 20 | 0.07163 | 0.0673 | 0.0245 | 0.0245 | 0.585 |

From the results above, one sees that increasing the angle, θ, results in smaller values of max pₙ while maintaining the same values for max qₙ. The result is a stiffening of the cutting edge as θ increases. In addition, the size of biopsy sample recess, S, decreases as θ increases.

In the examples given, the value of rᵢ was zero which brings the depth of the grind to the centerline of the inner cannula. Selecting values of rᵢ greater than zero produces a grind which is above the centerline of the inner cannula which results in a biopsy needle point which is more resistant to bending.

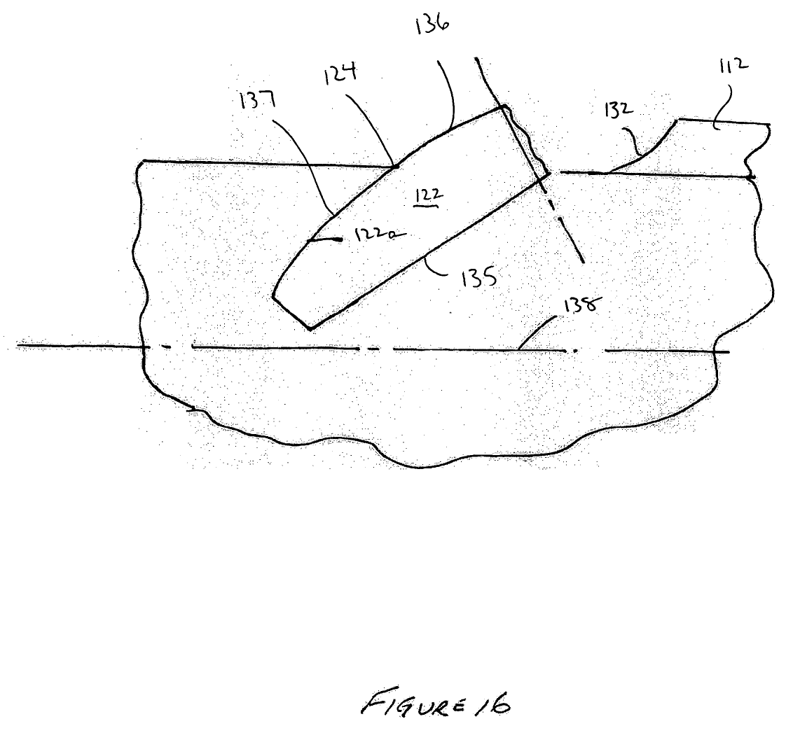

For comparison purposes, the pₙ vs. iₙ results for θ=10° and θ=20° were plotted in FIGS. 11 and 15, respectively. The same scale was used in the x and y directions in order to show the actual shape of the cutting edge on the inner cannula of the biopsy needles and to show the change in the shape of the cutting edges when the value of θ was changed from 10° to 20°. Considering the results for the 18 gage biopsy needle, the 18 GA pₙ vs. iₙ curve is the center curve on FIGS. 11 and 15. The value of pₙ for the θ=10° 18 GA needle is given on the interp (t, iₙ18, pₙ18, iₙ18) axis. The value of iₙ is given on the iₙ 18 axis. From FIG. 11, for θ=10°, the length of the cutting edge is 0.14 inches. Since the cutting edge curve is symmetrical about the vertical axis, the width of the large end of the cutting edge is 2×0.0245=0.049″ which is the OD of the inner cannula. From FIG. 15, for θ=20°, the length of the cutting edge is 0.067″ and the width of the large end of the cutting edge is 0.049″. Since the width of the large end is the same for both θ=10° and 0=20°, and the length of the cutting edge for θ=20° is only about half of that for the θ=10° design, changing the angle from 10° to 20° significantly increases the bending strength of the cutting edge.

Note that in the examples selected herein, the grinding depth was halfway through the inner cannula (i.e., rᵢ=0). It is, however, contemplated, other grinding depths may be used to produce the cutting edge desired by selecting a value of rᵢ between zero and the outer radius of the cannula, rₒ.

In operation, and as briefly discussed above, the biopsy needle 10 is inserted to the depth required and the outer cannula 12 is withdrawn and cocked. The portion of the tissue to be excised is in the region of the biopsy sample recess 22. When the outer cannula 12 is fired, the outer cannula 12 moves toward the distal tip 20 of the inner cannula 14. The portion of the tissue protruding into the biopsy sample recess 22 is cut off by the two cutting edges 24, 32 and is collected in the biopsy sample recess 22.

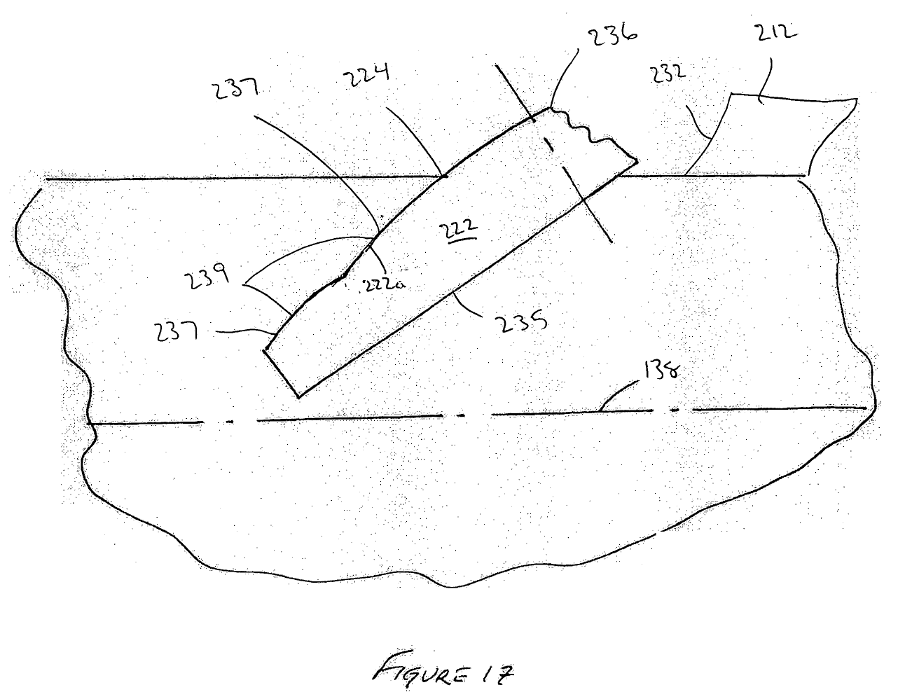

Referring to FIGS. 16 and 17, that the sharpness of the cutting edges 124, 132, 224, 232 may be increased by “hollow-grinding” the cutting edges 124, 132, 224, 232. This can be achieved on the inner cannula cutting edge 124, 224 by using a grinding wheel 136, 236, having opposed upper and lower grinding surfaces 135, 235, 137, 237, wherein the upper grinding surface(s) 137, 237 is convex to cut the recess 122, 222 and form a concave forward wall 122a, 222a (and in the case of the embodiment shown with reference to FIG. 17, having multiple convex surfaces 237 to produce multiple concave surfaces 239 along the forward wall 222a of the recess 222). The convex surface(s) 137, 237 on the grinding wheel 136, 236 will produce a concave, “hollow-ground” edge on the cutting edge 124, 224 of the inner cannula 114, 214. The same result can be achieved on the outer cannula 112, 212.

Due to the cutting rather than tearing operation, biopsy samples cut with the present biopsy needle should produce less trauma to the patient.

While the preferred embodiments have been shown and described, it will be understood that there is no intent to limit the invention by such disclosure, but rather, is intended to cover all modifications and alternate constructions falling within the spirit and scope of the invention.

1. A biopsy needle, comprising:
an outer cannula;
an inner cannula including a distal end and a proximal end, the outer cannula being shaped and dimensioned to closely circumscribe the inner cannula for movement relative thereto;
the inner cannula further including a sample recess at its distal end, the sample recess including a U-shaped cutting edge defined by
pₙ=xₙ cos θ
qₙ=xₙ sin θ
where,
θ is an angle at which the cutting edge is formed.
xₙ is an actual length position along the sample recess,
pₙ is a length position of the sample recess relative to the cutting edge along a line substantially parallel to an inner cannula longitudinal axis, and
qₙ is a depth position of the sample recess relative to an apex of an arc defined by a cutting edge.

2. The biopsy needle according to claim 1, wherein the maximum values for xₙ, pₙ, qₙ are as follows:
$$\begin{matrix}max {x}_{n} = \frac{{r}_{o} - {r}_{i}}{sin θ} \\ max {p}_{n} = \frac{{r}_{o} - {r}_{i}}{tan θ} \\ max {q}_{n} = {r}_{o} - {r}_{i}\end{matrix}$$
where,
rₒ is a radius of the inner cannula, and
rᵢ is a distance a base of the sample recess is from a centerline of the inner cannula.

3. The biopsy needle according to claim 2, wherein θ is between approximately 10° and approximately 20°.

4. The biopsy needle according to claim 3, wherein rₒ is approximately 0.0175″ and approximately 0.0245″.

5. The biopsy needle according to claim 2, wherein θ is approximately 20°.

6. The biopsy needle according to claim 5, wherein to is approximately 0.0175″ and approximately 0.0245″.

7. The biopsy needle according to claim 2, wherein iₙ, a distance from a centerline of a transverse cross section of the inner cannula to an edge of the cutting edge at an outer surface of the inner cannula is:
iₙ=±\sqrt{(2r₀−xₙ sin θ)xₙ sin θ}

8. The biopsy needle according to claim 1, wherein the cutting edge has a concave hollow-ground edge.

9. The biopsy needle according to claim 8, wherein the cutting edge has a plurality of concave hollow-ground surfaces.

10. An inner cannula of a biopsy needle manufactured in accordance with the method comprising the following steps:
setting a grinding wheel at an angle θ, relative to a longitudinal axis of the inner cannula;
grinding the inner cannula with the grinding wheel to produce a sample recess having a cutting edge at an outside surface of the inner cannula as the grinding wheel moves through the inner cannula in a direction transverse to the longitudinal axis of the inner cannula, wherein, the cutting edge is defined by
pₙ=xₙ cos θ
qₙ=xₙ sin θ
where,
θ is an angle at which the cutting edge is formed.
xₙ is an actual length position along the sample recess,
pₙ is a length position of the sample recess relative to the cutting edge along a line substantially parallel to an inner cannula longitudinal axis, and
qₙ is a depth position of the sample recess relative to an apex of an arc defined by a cutting edge.

11. The biopsy needle according to claim 10, wherein the maximum values for xₙ, pₙ, qₙ are as follows:
$$\begin{matrix}max {x}_{n} = \frac{{r}_{o} - {r}_{i}}{sin θ} \\ max {p}_{n} = \frac{{r}_{o} - {r}_{i}}{tan θ} \\ max {q}_{n} = {r}_{o} - {r}_{i}\end{matrix}$$
where,
rₒ is a radius of the inner cannula, and
rᵢ is a distance a base of the sample recess is from a centerline of the inner cannula.

12. The biopsy needle according to claim 11, wherein θ0 is between approximately 10° and approximately 20°.

13. The biopsy needle according to claim 12, wherein r₀ is approximately 0.0175″ and approximately 0.0245″.

14. The biopsy needle according to claim 11, wherein θ is approximately 20°.

15. The biopsy needle according to claim 14, wherein r₀ is approximately 0.0175″ and approximately 0.0245″.

16. The biopsy needle according to claim 11, wherein iₙ, a distance from a centerline of a transverse cross section of the inner cannula to an edge of the cutting edge at an outer surface of the inner cannula is:
iₙ±√ (2r₀=xₙ sin θ)xₙ sin θ)}

17. The biopsy needle according to claim 10, wherein the grinding wheel has opposed upper and lower grinding surface and the upper grinding surface is convex.

18. The biopsy needle according to claim 17, wherein the upper grinding surface has multiple convex surfaces.
